(19) United States
(12) Patent Application Publication
(10) Pub. No.: US 2022/0257115 A1
(43) Pub. Date: Aug. 18, 2022

(54) AN AUTOMATED SLIT LAMP WITH COMPUTER PARTS AND A METHOD OF EYE SCREENING USING THE SAME

(71) Applicant: NATIONAL UNIVERSITY OF SINGAPORE, Singapore (SG)

(72) Inventors: Muhammad Azri Bin RAZALI, Singapore (SG); Victor Teck Chang KOH, Singapore (SG)

(21) Appl. No.: 17/625,339

(22) PCT Filed: Jul. 7, 2020
(86) PCT No.: PCT/SG2020/050387

§ 371 Date: Jan. 7, 2022

(30) Foreign Application Priority Data
Jul. 8, 2019 (SG) 10201906314P

(51) Int. Cl. A61B 3/135; A61B 3/113; A61B 3/14; G16H 30/20; G16H 50/20

(52) U.S. Cl. CPC A61B 3/135; A61B 3/113; G16H 50/20; G16H 30/20; A61B 3/14

(57) ABSTRACT

An automated slit lamp with computer program parts, comprising imaging optics, imaging optics control, microcontroller with microcomputer interface, first computer program module for automated movements and automatic image acquisition, lighting optics, lighting optics control, and second computer program module for eye screening. A method of eye screening using the automated slit lamp with computer program parts is also provided.

### TECHNICAL FIELD

The present disclosure generally relates to a medical device, and more specifically to an automated slit lamp with computer program parts and a method of eye screening using the same.

### BACKGROUND

Slit lamp is a medical device commonly used for eye examination to facilitate diagnosis of various eye conditions. The existing slit lamp must be operated by a skilled physician.

Nowadays, due to insufficient resources for eye care, late diagnosis and suboptimal outcome of preventable vision diseases occurs quite often.

There is a need to provide a diagnosis device with image capturing and grading that would enable primary care practitioners to perform eye screening for major eye diseases.

### SUMMARY

In order to provide primary health care practitioners the ability to perform eye screening for various eye diseases while further providing them a grading or a report that would be a basis for determining whether or not a patient should be referred to an ophthalmologist for further examinations or treatment, the present disclosure provides an automated slit lamp with computer program parts and a method of eye screening using the same.

The present disclosure will enable general practitioners to screen and handle eye diseases. For countries with an aging population, the present disclosure will solve the economic burden healthcare has as a result of the aging population. Instead of eye screening being a test that only skilled ophthalmologists may perform, the present disclosure provides a system that may be operated by any primary care giver, thereby significantly reducing waiting time until such screening may be performed, and enabling a patient in need to receive the appropriate treatment within a shorter time period, and preferably at an early stage of the developing eye disease.

In one aspect, the present disclosure provides an automated slit lamp with computer program parts comprising:

imaging optics comprising an optical system and an imaging device, the imaging optics configured to automatically acquire an image of an eye of a patient:

imaging optics control connected to the imaging optics and configured to control position of the imaging optics:

microcontroller comprising a microcomputer interface configured to instruct and control automated movement of the imaging optics control;

a computer program module configured to provide instructions to the microcontroller thereby effecting the automated movements of the imaging optics control and automatic image acquisition;

lighting optics comprising illumination sources, the lighting optics configured to direct light from the illumination sources towards the eye of the patient; and

lighting optics control connected to the lighting optics configured to control illumination of the eye of the patient during an eye screening.

Optionally, the automated slit lamp system may further comprise a second computer program module configured for eye screening.

In some embodiments, the imaging optics may comprise a plurality of objective lens and a plurality of tube lens.

In some embodiments, the imaging optics control may be a 3-axis control, comprising:

a lead screw with roller on rails for z axis and y axis;

timing belts with rollers on rails for x axis;

at least one stepper motor for controlling movement of imaging optics along x axis, y axis and z axis, and

a stepper motor driver for controlling the at least one stepper motor.

In some embodiments, the first computer program module comprises a computer program for pupil detection and iris detection.

In some embodiments, the lighting optics may comprise:

a LED light source;

a collector lens acting as a LED collector;

an adjustable mask for a spot or a slit for profiling light for an eye examination:

a field lens for creating a magnified intermediary image of aperture stop;

a condenser lens for condensing or collimating light, and

a beam splitter for making light coaxial with the imaging device and removing glares from an acquired image.

In some embodiments, the lighting optics father comprises a field diaphragm locating between the collector lens and the field lens.

In some embodiments, the lighting optics further comprises a brightness control aperture.

In some embodiments, the lighting optics further comprises a lens for posterior imaging.

In some embodiments, the lighting optics control comprises a stepper motor to rotate slit lighting at different degrees for controlling the lighting optics.

In some embodiments, the second computer program module for eye screening comprises a computer program for eye screening.

In another aspect, the present disclosure provides a method of eye screening using an automated slit lamp with computer program parts, comprising:

conducting different eye examinations for detecting different eye conditions on each eye of a patient, one by one, by using the automated slit lamp with computer program parts disclosed hereinabove;

acquiring images from the different eye examinations;

processing the images by using the computer program parts to obtain gradings for the different eye examinations; and

generating a report of eye conditions that the patient is suspected of having by consolidating all the gradings from the different eye examinations.

In some embodiments, the different eye conditions may be cataract, glaucoma. Age Related Macular Degeneration, Diabetic Retinopathy or other eye conditions.

In some embodiments, glaucoma is Narrow Angle Glaucoma or Open Angle Glaucoma.

In some embodiments, the method may further comprise determining whether the images are gradable before processing the images to obtain gradings.

In some embodiments, the method may further comprise adjusting focus, centering the images and positioning light at a correct position for adjusting the images as gradable images before processing the images to obtain gradings.

### BRIEF DESCRIPTION OF THE DRAWINGS

Some non-limiting exemplary embodiments or features of the disclosed subject matter are illustrated in the following drawings.

In the drawings:

FIG. 1 is a schematic illustration of the automated slit lamp with computer program parts of the present disclosure;

FIG. 2 is a schematic illustration of the imaging optics control of the automated slit lamp of the present disclosure;

FIG. 3 is a schematic illustration of the imaging optics of the automated slit lamp of the present disclosure;

FIG. 4 is a schematic illustration of lens simulation of Kohler illumination using LED;

FIG. 5 is a schematic illustration of lens simulation of Kohler illumination with a beam splitter:

FIGS. 6A-6C are schematic illustrations of the lighting optics control of the automated slit lamp of the present invention;

FIG. 7 is a schematic flow chart illustrating the general process of the method of eye screening of the present disclosure;

FIG. 8 is a schematic flow chart illustrating the examination process of cataract, Narrow Angle Glaucoma, Open Angle Glaucoma, Age Related Macular Degeneration, and Diabetic Retinopathy by using the method of eye screening of the present disclosure;

FIG. 9 is a picture showing how an image obtained in an eye examination for narrow angle glaucoma is segmented:

FIG. 10 is a set of images showing some narrow angle examples with different Van Herick gradings, detected by the method of eye screening of the present disclosure;

FIG. 11 shows images for open angle glaucoma showing an increased cup to disc ratio, detected by the method of eye screening of the present disclosure:

FIG. 12 shows images for Age Related Macular Degeneration, detected by the method of eye screening of the present disclosure;

FIG. 13 is an image for Diabetic Retinopathy, detected by the method of eye screening of the present disclosure:

FIGS. 14A-14B depict a flow chart illustrating a method for anterior segment imaging for examining Narrow Angle Glaucoma and Cataract; and

FIGS. 15A-15B depict a flow chart illustrating a method for posterior segment imaging for examining Open Angle Glaucoma. Age Related Macular Degeneration and Diabetic Retinopathy.

### DETAILED DESCRIPTION

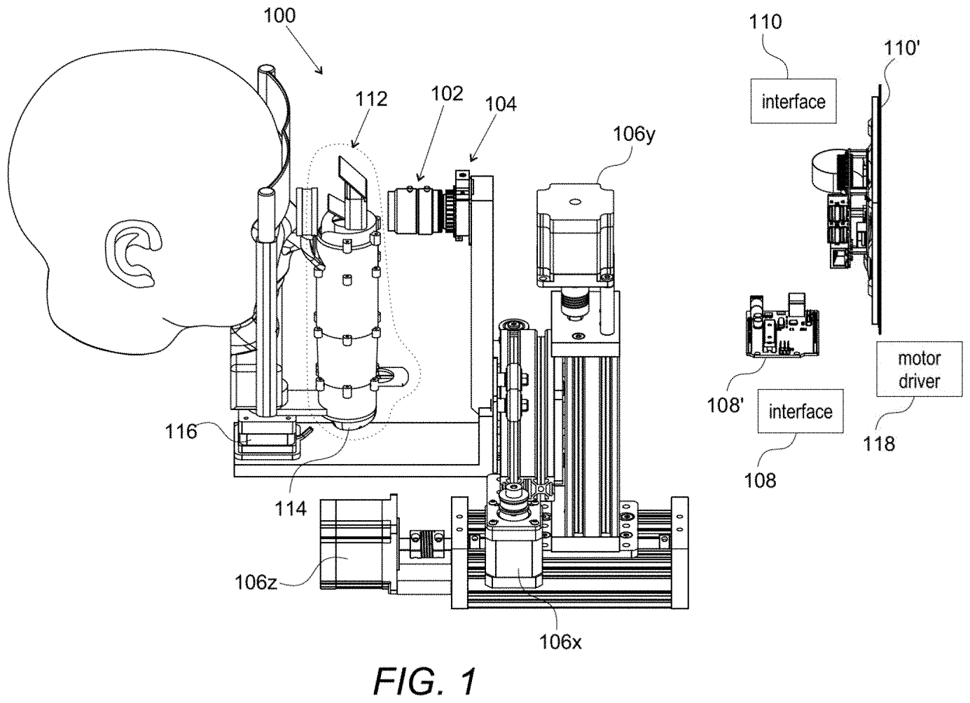

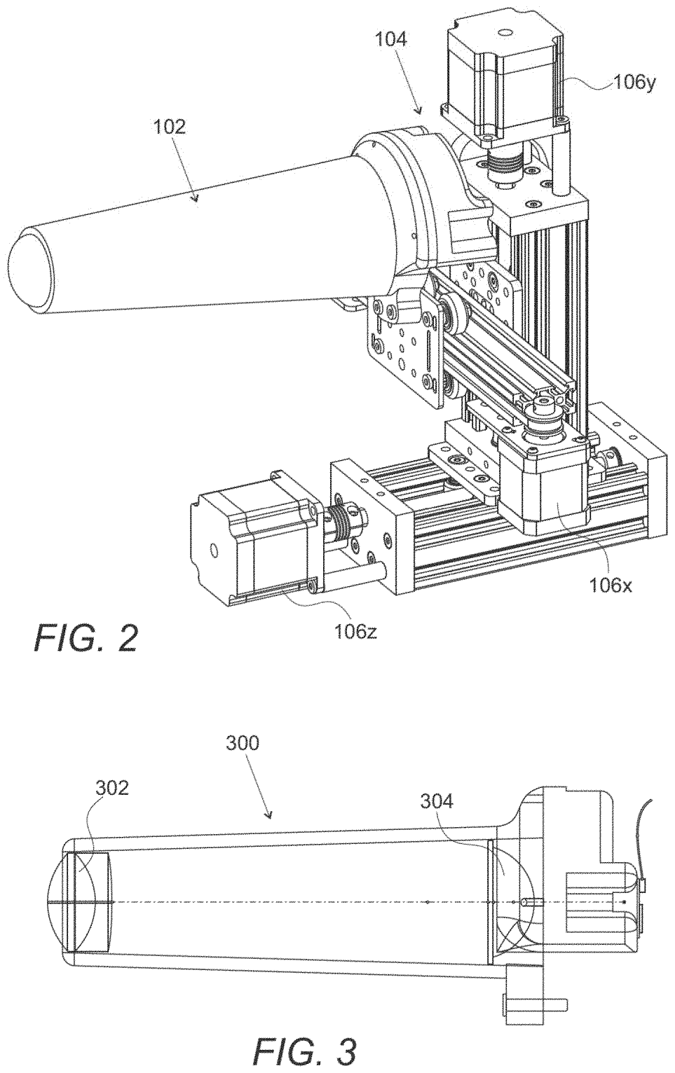

With reference to FIGS. 1 and 2, the present disclosure provides an automated slit lamp with computer program parts system 100, comprising imaging optics 102 configured to direct light towards an imaging device or camera 104, e.g., a CMOS sensor, imaging optics control 106 configured to control position of the imaging optics 102, microcontroller 108′ with microcomputer interface 110, a first computer program module for automated movements and automatic image acquisition, lighting optics 112 configured to direct light from at least one illumination source 114, lighting optics control 116 configured to control position of the lighting optics 112, and a second computer program module for eye screening.

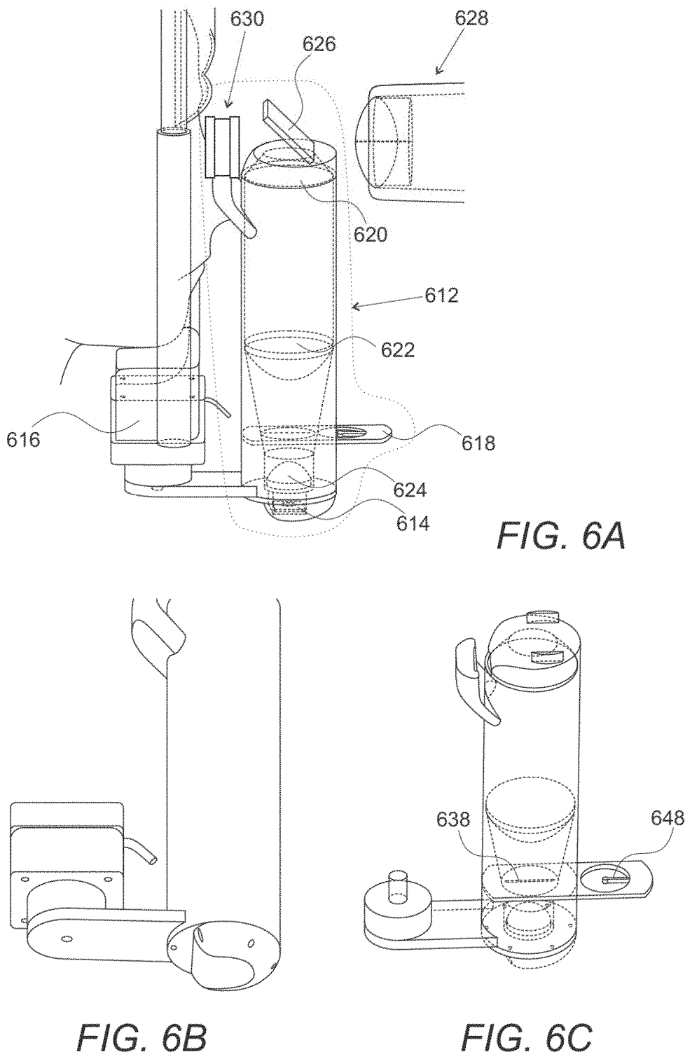

In accordance with some embodiments, the imaging optics 102 consists of multi-element lenses. The imaging optics 102 may be automatically manipulated by the imaging optics control 106. According to some embodiments, the lighting optics 112 use a method of illumination known as Kohler Illumination. Kohler Illumination may control the shape of the light illuminated by at least one illumination source 114 that is located proximate to the lighting optics 112. Köhler Illumination may also dictate how bright the light is by adding masks (e.g., mask 618 in FIG. 6A) and dictating the size of the aperture (not shown). Kohler Illumination may also help to ensure that the light illuminating the specimen is collimated. i.e. that no image of the light source, e.g., illumination source 114 appears on the specimen, which in this case is an eye of a patient.

In some embodiments, microcomputer 110′ through microcomputer interface 110 may determine information with respect to an acquired image, for example, how much focus is required to acquire a high-quality image. The microcomputer 110′ sends instructions to microcontroller 108′. In some embodiments, microcontroller 108′ may handle low level controlling, for example, microcontroller 108′ may determine in which direction to move the motors, e.g., motors 106, which comprise motor 106x configured to move the imaging optics 102 along the x-axis, motor 106y configured to move the imaging optics 102 along the y-axis, and motor 106z configured to move imaging optics 102 along the z-axis. Microcontroller 108′ may further determine the amount of movement of motors 106, which move imaging optics 102, and may determine the speed at which the motors are to be moved. Microcontroller 108′ interfaces with motor driver 118, which has three roles—it receives information regarding the final position of imaging optics 102, the direction at which to move the at least one stepper motor 106 and 116 and whether the at least one stepper motor 106 and 116 should stop moving or keep on moving. The stepper motors 106 and 116, e.g., motors 106x, 106y. 106z and 116 are executing the instructions provided by motor driver 118, which received the instructions from microcontroller 108′ via microcontroller interface 108, which received instructions from microcomputer 110′ via microcomputer interface 110.

The microcontroller 108′ with microcontroller interface 108 may instruct the necessary manipulation as dictated by the first computer program module. The first computer program may also provide instructions relating to the lighting optics control 106 so as to enable the use of light in the performance of the eye examination and acquisition of images which are processed by the second computer program which may also perform an eye screening.

According to FIG. 3, the imaging optics of the automated slit lamp with computer program parts 100 of the present invention may comprise a plurality of objective lens 302 and a plurality of tube lens 304. Additional objective lenses and tube lenses may reduce footprint of optical devices or may condense more light in near camera for short exposure needs since more lenses could prevent vignetting of light rays. In one embodiment, the imaging optics 102 may comprise one objective lens 302 and one tube lens 304.

In some embodiments, the objective lens, e.g., objective lens 302 may have a focal length of 50 mm in order to ensure a working distance of 75 mm from an eye of a patient to the objective lens 302. The objective lens 302 may be achromatic in order to reduce chromatic aberrations as much as possible. The objective lens 302 may have a diameter ranging from 40 mm to 50 mm, and preferably the objective lens may have a diameter of 50 mm.

In accordance with some embodiments, the objective lens in the imaging optics 102 may magnify an image by two folds at around 128-150 mm behind the objective lens. e.g., objective lens 302. The second lens may condense the magnified image into the camera image sensor 104 to fully fill the sensor area so that the camera 104 may capture the full 17 mm area of the eye to be in focus. The camera lens, of focal length 3.04 mm, may preferably be adjusted to around 3.093 mm away to focus the virtual magnified image which gives around 10× magnification.

In some embodiments, the imaging optics control of the automated slit lamp with computer program parts 100 of the present disclosure can be a 3-axis control, comprising a lead screw with roller on rails for z axis and y axis, a timing belt with rollers on rails for x axis, one or more stepper motors (e.g., NEMA 17 stepper motor) 106x, 106y and 106z for controlling x axis, y axis and z axis, respectively, and one or more stepper motor driver 118 (e.g., DM542T stepper motor driver) for controlling the stepper motors 106.

In some embodiments, the microcontroller 108′ with microcomputer interface 110 of the automated slit lamp with computer program parts 100 of the present disclosure may comprise microcontroller module, microcomputer module and infrared distance sensor module, wherein, the microcomputer 110′ (e.g., raspberry pi) sends commands to the microcontroller 108′ (e.g., Arduino) to control the stepper motors 106; the microcomputer 110′ may receive inputs from object detection, for example, size of iris, sclera and pupil, and may use it to determine distance (obtained through calibration) between the objective lens (e.g., objective lens 302) and a patient's eye and make finer adjustments of the stepper motors 106 for controlling y-axis. The results from the pupil detection may assist in correctly centering the camera 104 with respect to the pupil center by adjusting the stepper motors 106 for controlling x-axis and z axis. The microcomputer 110′ may then send commands to set the light optics 102 position depending on the eye examinations to be performed.

In some other embodiments, the microcontroller 108′ with microcomputer interface 110 may further comprise an infrared distance sensor (not shown) to aid in determining the distance between the objective lens 302 and a patient's face (and thus patient's eyes) and move stepper motors 106 for controlling y-axis using the microcontroller to an ideal position relative to the patient's eyes.

In some embodiments, the first computer program module for automated movements and automatic image acquisition of the automated slit lamp with computer program parts 100 of the present disclosure may comprise a computer program for pupil detection and iris detection. A compute stick, for example, neural compute stick may be used for running the computer program. The pupil and iris detected may be used to determine if the image is in focus. When everything is in place, a simple network may activate auto-capturing or auto-acquisition of an image based on the objects detected, focus and position of light. YOLOv3 algorithm may be used to train the computer program for detection. According to some embodiments, the same labeled images may be passed to additional or other object detection algorithms.

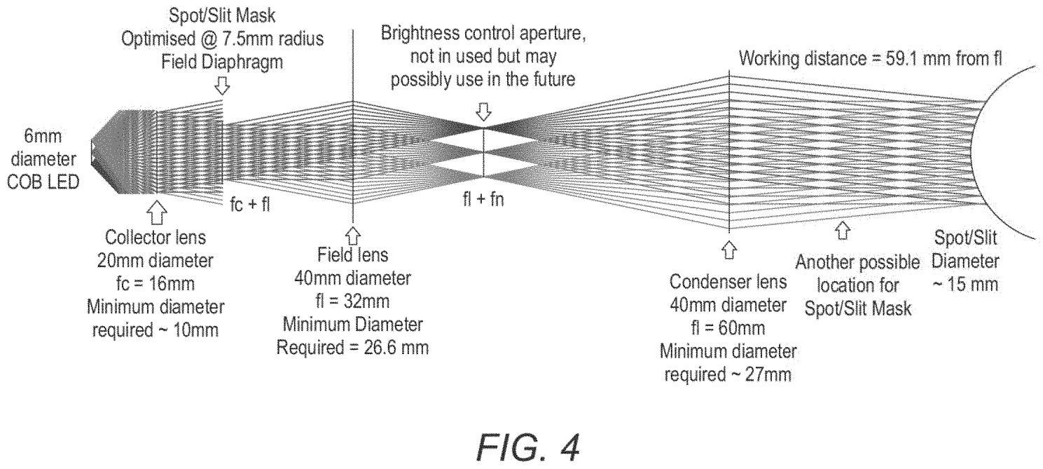

The lighting optics of the automated slit lamp with computer program parts 100 of the present disclosure may be implemented according to Kohler illumination method to achieve collimated light that does not form an image at the area of lighting. The lens simulation of Kohler illumination using an illumination source, e.g., a LED of the lighting optics 112 is illustrated in FIG. 4.

According to FIGS. 6A to 6C, the lighting optics 112 of the automated slit lamp with computer program parts 100 of the present disclosure may comprise a LED light source. e.g., light source 614, collector lens. e.g., collector lens 624 acting as a LED collector, adjustable mask. e.g., mask 618 for a spot or a slit for profiling light for eye examination, field lens. e.g., field lens 622 for creating a magnified intermediary image of aperture stop, condenser lens, e.g., condenser lens 620 for condensing or collimating light, beam splitter. e.g., beam splitter 626 for making light coaxial with camera 104 and removing glares from the acquired image, and stepper motor, e.g., stepper motor 616 that rotates lighting optics 612.

In some embodiments, the LED light source 614 may be a Chip On Board LED, also known as COB LED. In some embodiments, the COB LED may have a diameter of 6 mm. In other embodiments, the COB LED may have a voltage of 9 V and may only require a current of 700 mA to achieve a luminous flux of around 400 lumens, which is equivalent to a 10 w halogen lamp. Light source 614 may be any type of light source, besides an LED, however, the light source must be positioned 9 mm away from the back of the collector lens 624 (which defines the back focal length).

In some embodiments, the collector lens 624 may have a diameter ranging from 10 mm to 20 mm, preferably 20 mm.

In some embodiments, the lighting optics 612 may comprise more than one adjustable mask 618. In some embodiments, mask 618 may comprise a slit mask 638 and a fundus mask 648 (FIG. 6C), which may be interchangeable. Masks 638 and 648 or any other type of masks may be switched manually, or may be switched or interchanged automatically, e.g., by a motor, such to be operated and controlled by a microcontroller, such as microcontroller 108′.

In some embodiments, the field lens 622 may have a diameter ranging from 26.6 mm to 40 mm, preferably 40 mm. In some embodiments, the field lens 622 may have a focal length (fl) of 32 mm.

In some embodiments, the condenser lens 620 may have a diameter ranging from 27 mm to 40 mm, preferably 40 n. In some embodiments, the condenser lens 620 may have a focal length (fl) of 60 mm, preferably, 59.1 mm. The condenser lens 620 may ensure the focused light is collimated and falls onto a patient's eye optimally within the working distance.

In some embodiments, the beam splitter 626 may comprise polarized prisms to make light coaxial with camera 104 (FIG. 1) and to remove glares from acquired images by cross polarization between the camera 104 and the light optics 612 (or 112. FIG. 1).

In some embodiments, the lighting optics 612 may further comprise a field diaphragm located at the focal length between a collector lens 624 and a field lens 622 for shaping light to ensure that the field of illumination is well controlled for optimal lighting. In some embodiments, the field diaphragm may have a radius of 7.5 mm.

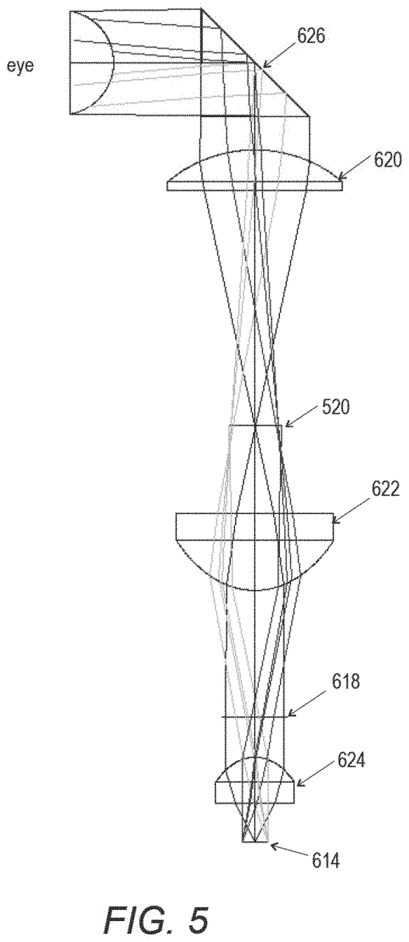

In some embodiments, the lighting optics 612 may further comprise a brightness control aperture 520 (FIG. 5), for example, aperture diaphragm, located at focal length between a field lens 622 and a condenser lens 620 for controlling brightness.

In some embodiments, the lighting optics 612 may further comprise a lens for posterior imaging 630, e.g. 78D lens, for fundus imaging.

In some embodiments, the lighting optics control. e.g., lighting optics control of the automated slit lamp with computer program parts 100 of the present disclosure may comprise a stepper motor 116 to rotate slit lighting at different degrees for controlling the lighting optics 612. The center of rotation of motor 116 may be at the front of a patient's eye. In some embodiments, the lighting optics control may comprise a single stepper motor 116.

According to some embodiments, OpenCV, the variance of Laplacian method may be used to obtain a value, which may be used to determine if the obtained picture is in focus or not. This may require calibration on what values constitute a blur image or a sharp image. It may be accomplished by convolving input image with the Laplacian operator and computing the variance afterwards. If the variance falls below a predefined “blur” threshold, the picture may be deemed as blurred and the motor 116 may keep on moving until the picture is in focus. i.e., the variance is above the “blur” threshold.

In some embodiments, the second computer program module for eye screening of the automated slit lamp with computer program parts 100 of the present disclosure may comprise a computer program for eye screening. A compute stick, for example, neural compute stick, may be used for running the computer program. YOLOv3 or SSD or Faster R-CNN or any other algorithms may be used to train the computer program for screening. To achieve a complete screening, each of the examination's results may be collated, processed and graded using each of their grading method using image processing. The second computer program module for eye screening may generate a report to determine whether a patient should be referred to an ophthalmologist or not. The second computer program module for eye screening may be trained based on an ophthalmologist input on whether it is a referable case based on the different examined conditions.

In accordance with some embodiments, the present disclosure may provide a method of eye screening with an automated slit lamp with computer program parts 100 described in the present disclosure, the method comprising conducting different eye examinations for different eye conditions on each of a patient's eyes, one by one, by using the automated slit lamp with computer program parts 100, obtaining images from the different eye examinations, processing the obtained images by using the computer program parts to obtain gradings for the different eye examinations, and generating a report of eye conditions that the patient is suspected of having by consolidating all the gradings from the different eye examinations.

FIG. 6B illustrates the swivel point of stepper motor 616 for the lighting optics 612, the swivel point adapted to ensure that the centre of rotation of the lighting optics 612 coincides with the centre of rotation of the patient's eye.

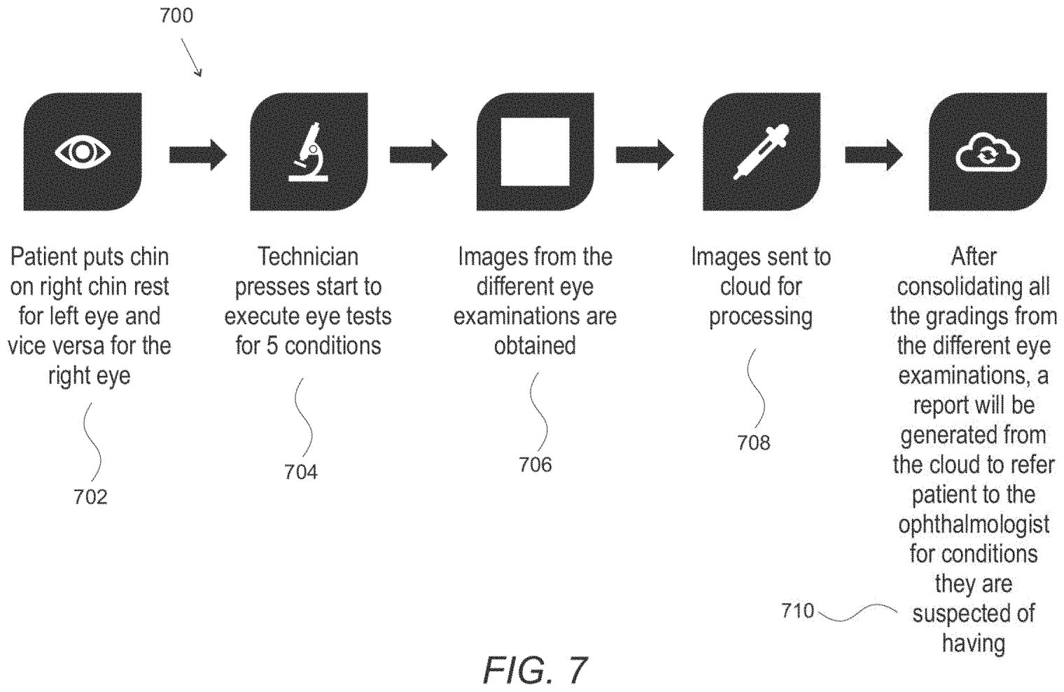

With reference to FIG. 7, a flow chart is depicted showing the general process of the method 700 of eye screening according to the present disclosure. In some embodiments, the method of eye screening of the present disclosure may be used to screen for cataract, glaucoma. Age Related Macular Degeneration, Diabetic Retinopathy and other eye conditions.

Method 700 may comprise a patient placing his chin on either the right chinrest for left eye examination or on the left chinrest for right eye examination 702. The technician may press start to execute eye tests for five different conditions 704. Images from the different eye conditions may be obtained 706, the obtained images may be sent to a cloud for processing 708, and after consolidating all the gradings from the different eye examinations, a report may be generated from the cloud to refer the patient to the ophthalmologist per conditions he is suspected as having 710.

Preferably, in some embodiments, the method of eye screening of the present disclosure may be used to screen for cataract, Narrow Angle Glaucoma. Open Angle Glaucoma, Age Related Macular Degeneration, and Diabetic Retinopathy.

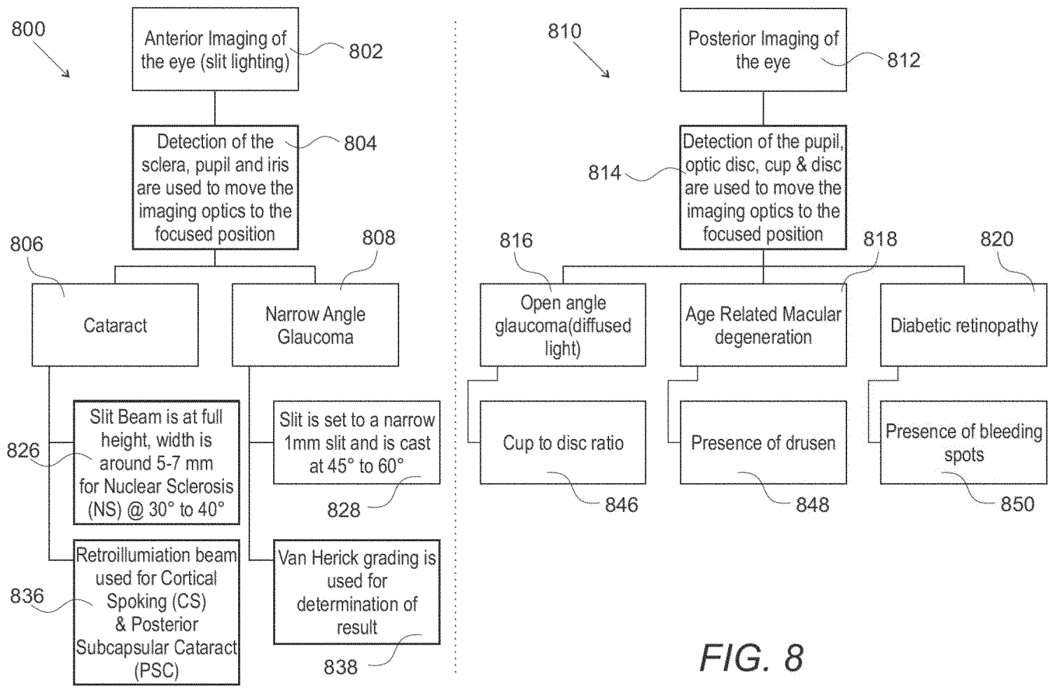

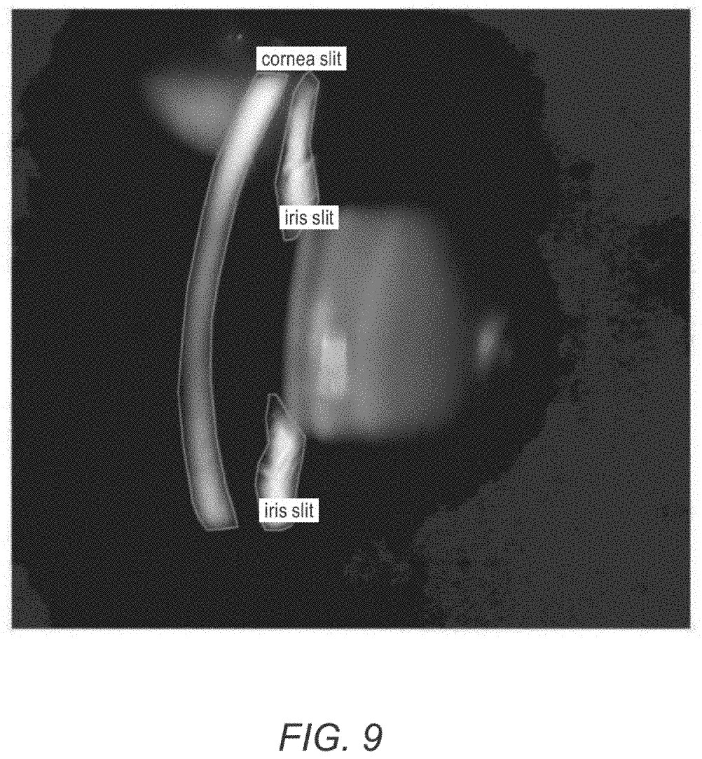

FIG. 8 is a schematic flow chart illustrating the examination process 800 of cataract. Narrow Angle Glaucoma, and examination process 810 of Open Angle Glaucoma. Age Related Macular Degeneration, and Diabetic Retinopathy by using methods of eye screening of the present disclosure.

In some embodiments, method 800 for examination process of cataract and Narrow Angle Glaucoma may comprise anterior imaging of an eye of a patient with slit lighting 802. Method 800 may comprise detecting of the sclera, pupil and iris for moving the imaging optics, e.g., imaging optics 102 to a focused position 804. In case of cataract 806, a slit beam is located at full height, width is around 5-7 mm for Nuclear Sclerosis at 30 to 40 degrees 826 and retroillumination beam may be used for Cortical Spoking & Posterior Subcapsular Cataract 836.

For Narrow Angle Glaucoma 808, slit may be set to a narrow 1 mm slit and in cast at 45 to 60 degrees 828 and Van Herick grading may be used for determination of the result 838.

In some embodiments, method 810 for examination process of Open Angle Glaucoma, Age Related Macular Degeneration, and Diabetic Retinopathy comprises posterior imaging of an eye of a patient 812 and detection of the pupil, optic disc. cup and disc may be used to move the imaging optics to a focused position 814. In case of Open Angle Glaucoma (diffused light) 816, a cup to disc ratio may be measured 846. In case of Age Related Macular Degeneration 818, presence of drusen may be determined 848. And in case of Diabetic Retinopathy 820, presence of bleeding spots may be detected 850.

Screening for Cataract

There are three types of cataract, which may be screened for by using the method of eye screening with an automated slit lamp with computer program parts described in the present disclosure. The images obtained from an eye examination for cataract may be labeled using the computer program module for image segmentation by using for example Mask RCNN. Deeplab Xception and other methods.

The first type cataract—Nuclear sclerotic cataract (NS), comprises cloudiness of the nucleus, which is the central portion of the lens. This type of cataract is best graded with slit beam at 30 to 45 degree angle with respect to the cataract. In an early cataract, the central nucleus is clearer than the anterior and posterior embryonic layers of the lens.

The second type cataract—Cortical spoking cataract (CS) comprises swelling of the cortex causing spoke/wedge-like peripheral cloudiness. This type of cataract is best graded while visualized with retroillumination. It may be difficult to retroilluminate if there is a concurrent NS cataract. By WHO criteria, the lens area of the peripheral spoke like opacities is summed up.

The third type cataract—Posterior subcapsular cataract (PSC) comprises opacity in the posterior capsule of the lens, often seen in younger individuals, steroid users, and patients suffering from diabetics. Like CS. PSC may be difficult to retroilluminate if there is a dense concurrent NS cataract. WHO criteria is used, graded on vertical height (in mm).

Screening for Narrow Angle Glaucoma

The images obtained from an eye examination for narrow angle glaucoma may be labeled to detect cornea slit and iris slit using the computer program module for image segmentation by using, for example. Mask RCNN. Deeplab Xception and other methods. Van Herick grading may be used to access peripheral anterior chamber depth at the slit lamp. This is a quick way to gauge the width of the angle that involves bringing the slit beam at an angle of 60 degrees onto the cornea just anterior to the corneal limbus (the border of the cornea and the sclera). A physician may estimate the anterior chamber depth between the peripheral iris and the corneal endothelium and compares it to the overall thickness of the cornea. Hereinafter is a guide to the Van Herick findings:
Grade 0 is used to describe peripheral iridocorneal contact.
Grade I is a space between the iris and corneal endothelium of less than one fourth the corneal thickness.
Grade II is the space of between one-fourth and half the thickness of the cornea.
Grade III is considered a non-occludable angle, with the distance being equal to or greater than half the corneal thickness.

FIG. 9 illustrates a picture showing how an image obtained in an eye examination for narrow angle glaucoma is segmented. The cornea slit and iris slit are illustrated.

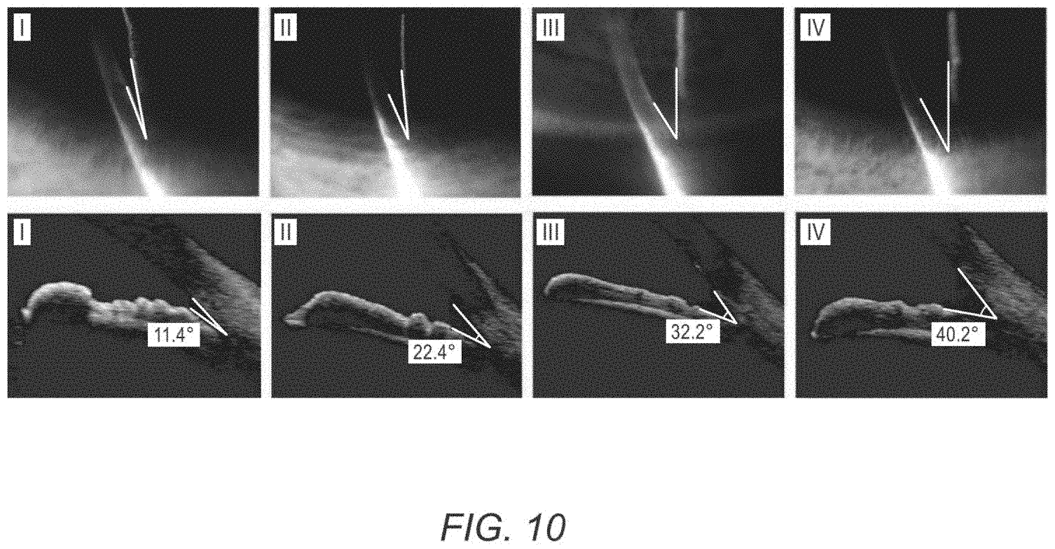

In FIG. 10, several narrow angle examples detected by the method of eye screening of the present disclosure are provided to illustrate how the Van Herick grading is used.

Screening for Open Angle Glaucoma

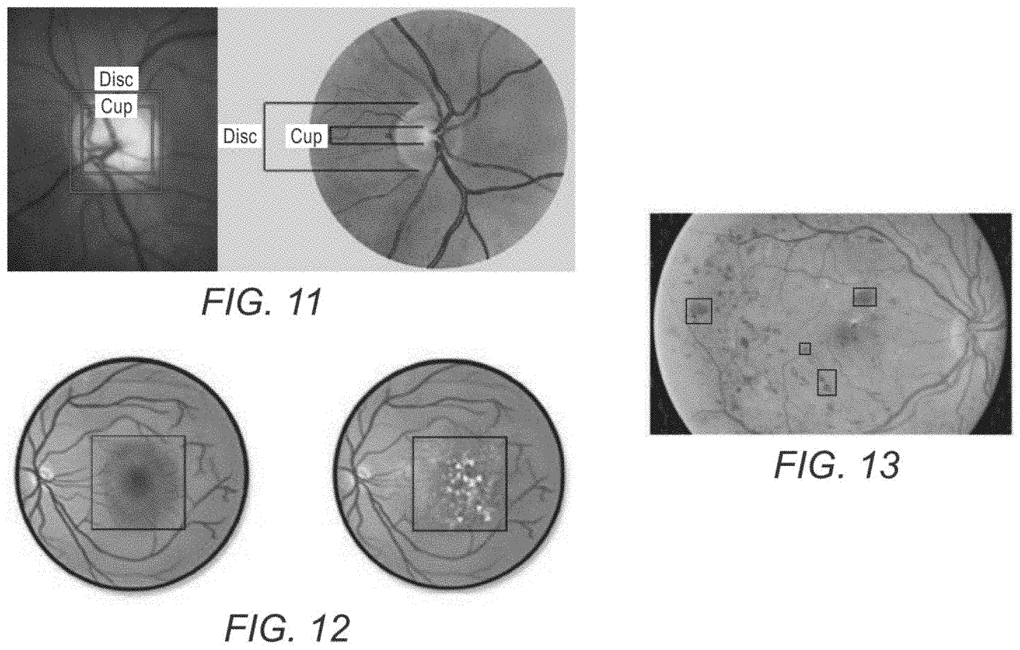

The images obtained from an eye examination for open angle glaucoma may be processed by the computer program module using Yolov3 or SSD or other algorithms to detect sizes of a cup and a disc and the ratio between the two sizes may determine a cup to disc ratio. An increased cup to disc ratio indicates that there may be presence of open angle glaucoma. FIG. 11 illustrates images for open angle glaucoma showing an increased cup to disc ratio, detected by the method of eye screening of the present disclosure.

Screening for Age Related Macular Degeneration (AMD)

The images obtained from an eye examination for AMD may be processed by the computer program module to box all the features of AMD detected and grade severity. Early screening of AMD usually are the amount of drusen visible on retina. FIG. 12 illustrates images for Age Related Macular Degeneration, detected by the method of eye screening of the present disclosure.

Screening for Diabetic Retinopathy

The fundus images obtained from an eye examination for diabetic retinopathy may be processed by the computer program module to search for neovascularization, micro aneurysms, exudates, edema and “cotton wool” spots, box the features detected above, and grade severity. FIG. 13 illustrates an image for Diabetic Retinopathy, detected by the method of eye screening of the present disclosure.

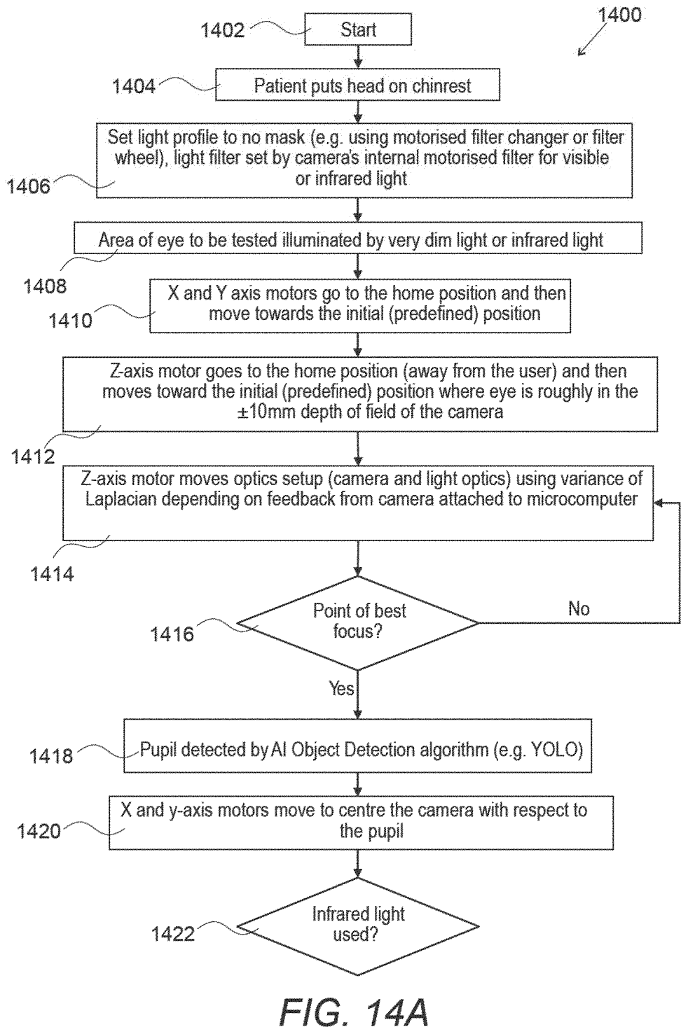

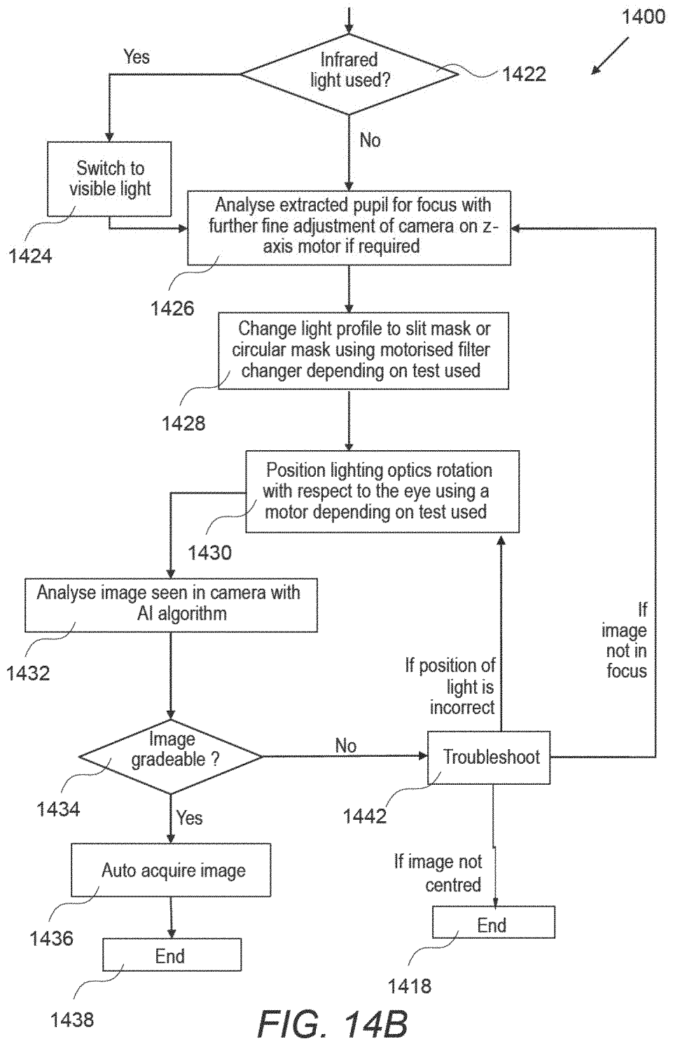

Reference is now made to FIGS. 14A-14B, which depict a flow chart illustrating a method for anterior segment imaging for examining Narrow Angle Glaucoma and Cataract. AI based processors may determine whether an acquired image is gradeable, i.e., whether the image is in good quality and may be graded per different eye examinations. If an image is not gradeable, the reason may be determined (for example, the image is not in focus, positioning of light is incorrect, or image is not centred) and then system 100, specifically microcomputer 110′ may correct the problem causing the image to not be gradable, by performing the appropriate different steps in flowchart 1400.

According to some embodiments, method 1400 starts at 1402 after which a patient puts his head on an automated slit lamp with computer program parts system 100's chinrest at operation 1404. In case the right eye is to be examined, the patient puts his head on the left chinrest and vice versa.

In some embodiments, in operation 1406, the light profile is set to no mask (e.g., using a motorised filter change or filter wheel or manually) and light filter is set by the camera's internal motorised filter for visible or infrared light. Thereby, a very dim light or infrared light may illuminate the eye area in operation 1408. Method 1400 may further comprise operation 1410 in which X and Y axis motors go to the home position and then move towards the initial (predefined) position. In some embodiments, in operation 1412, Z-axis motor may go to the home position (away from the user) and may then move towards the initial (predefined) position where the eye of a patient is roughly in the ±10 mm depth of field of the camera. In operation 1414, Z-axis motor may move optics setup (camera and light optics) using variance of Laplacian depending on feedback from camera attached to the microcomputer.

In some embodiments, microcomputer may determine in operation 1416 whether or not the point is of best focus. If best focus is achieved, then in operation 1418 the pupil may be detected by AI Object Detection Algorithm (e.g., YOLO), and then in operation 1420 the x-axis and y-axis motors may move to center the camera with respect to the pupil. In case best focus is not achieved, then operation 1414 is repeated.

In some embodiments, after best focus is achieved, the microcomputer may determine whether infrared light is used, in operation 1422. If infrared light is used, the microcomputer may instruct system 100 to switch to visible light in operation 1424. If infrared light is not used, then operation 1426 is implemented. In operation 1426, the extracted pupil is analyzed for focus with further fine adjustments of camera on z-axis motor if required.

In some embodiments, microcomputer may change the light profile to a slit mask or circular mask with using a motorised filter changer depending on the test used in operation 1428. The slit mask or circular mask used may be of differing sizes.

In some embodiments, microcomputer may position lighting optics rotation with respect to the eye using a motor depending on test used in operation 1430.

According to some embodiments, method 1400 may further comprise operation 1432 in which the image seen in camera is analyzed with AI algorithm. Microcomputer may determine in operation 1434 whether the image is gradable. If the image is gradable, then the image is automatically acquired in operation 1436, and the process ends in 1438. However, if the image is not gradable, there is troubleshoot 1442. In case the position of light is incorrect, then operation 1430 is implemented. In case the image is not centered, then operation 1418 is implemented. And in case the image is not in focus, operation 1426 is implemented.

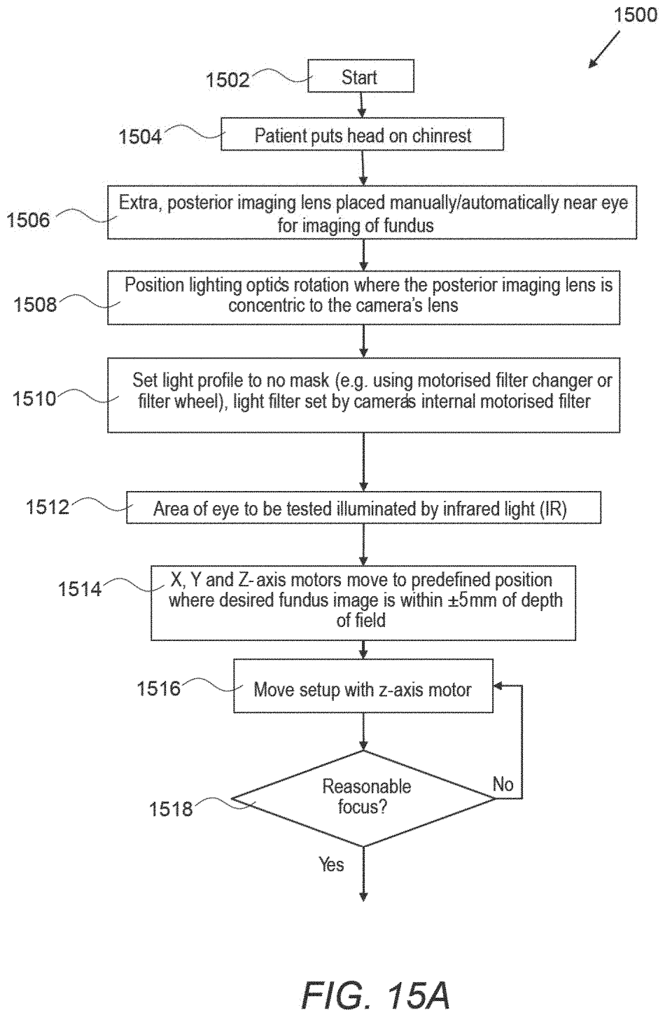

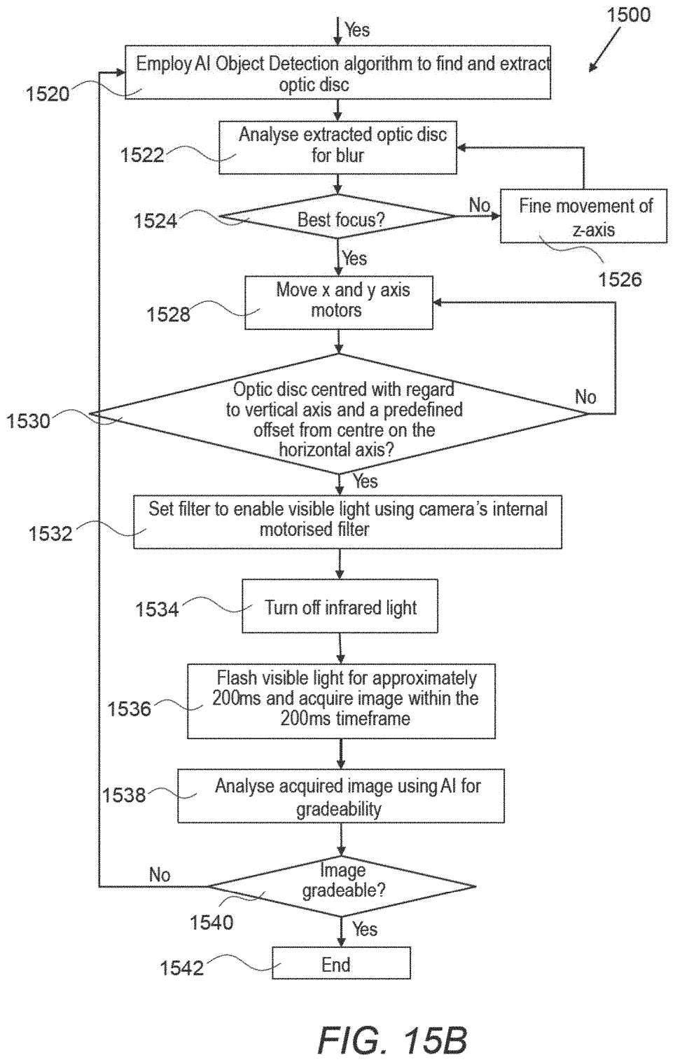

Reference is now made to FIGS. 15A-15B, which depict a flow chart illustrating a method for posterior segment imaging for examining Open Angle Glaucoma, AMD and Diabetic Retinopathy. AI based processors or microcomputers may determine whether an acquired image is gradeable, i.e., whether the image is in good quality and may be graded per different eye examinations. If an image is not gradeable, the reason may be determined (for example, the image is not in focus, positioning of light is incorrect, or image is not centred) and then system 100, specifically microcomputer 110′ may correct the problem causing the image to not be gradable, by performing the appropriate different steps in flowchart 1500.

According to some embodiments, method 1500 starts at 1502 after which a patient puts his head on an automated slit lamp with computer program parts system 100's chinrest at operation 1504. In case the right eye is to be examined, the patient puts his head on the left chinrest and vice versa. In operation 1506, an extra posterior imaging lens may be placed manually or automatically near an eye of the patient for imaging the fundus (this extra lens may be removed when acquiring anterior imaging and added when acquiring posterior imaging). In operation 1508, the lighting optics rotation may be positioned where the posterior imaging lens is concentric to the camera's lens. In operation 1510, the light profile is set to no mask, e.g., by using a motorised filter changer or filter wheel, and light filter may be set by the camera's internal motorised filter.

In operation 1512, the area of the eye that is to be tested may be illuminated with infrared light. Following operation 1512, operation 1514 may comprise X, Y and Z axis motors to move to predefined position where desired fundus image is within ±5 mm of depth of field. In operation 1516, Z-axis motor may move the setup. In operation 1518 the microcomputer may determine whether or not the focus achieved is reasonable. If it is not, then operation 1516 is repeated. If the focus is reasonable, then in operation 1520 AI Object Detection Algorithm is employed to find and extract the optic disc. In operation 1522 the extracted optic disc is analyzed for blur. Focus is again checked in operation 1524. If focus is not best, then Z axis is moved in fine movements, in operation 1526 and extracted optic disc is analyzed for blur in operation 1522. If best focus is achieved in operation 1524, then X and Y axis motors may be moved in operation 1528. In operation 1530, microcomputer may determine whether optic disc is centered with regard to vertical axis and a predefined offset from center on the horizontal axis. If optic disc is not centered, then operation 1528 is repeated. If the optic disc is centered, then in operation 1532 a filter is set to enable visible light using camera's internal motorised filter.

In some embodiments, method 1500 may further comprise operation 1534 in which infrared light is turned off. In operation 1536, visible light is flashed for approximately 200 ms and at least one image is acquired within the 200 ms timeframe. In operation 1538 the acquired image is analyzed using AI for gradeability. The microcomputer may determine whether the image is gradable in operation 1540. If it is not gradable, then operation 1520 is repeated. If the acquired image is gradable, the process ends at 1542.

While certain embodiments of the disclosed subject matter have been illustrated and described, it will be clear that the disclosure is not limited to the embodiments described herein. Numerous modifications, changes, variations, substitutions and equivalents are not precluded.

1. An automated slit lamp system with computer program parts, comprising:
imaging optics comprising an optical system and an imaging device, the imaging optics configured to automatically acquire an image of an eye of a patient;
imaging optics control connected to the imaging optics and configured to control position of the imaging optics;
microcontroller comprising a microcomputer interface configured to instruct and control automated movement of the imaging optics control;
a first computer program module configured to provide instructions to the microcontroller thereby effecting the automated movements of the imaging optics control and automatic image acquisition;
lighting optics comprising illumination sources, the lighting optics configured to direct light from the illumination sources towards the eye of the patient; and
lighting optics control connected to the lighting optics configured to control illumination of the eye of the patient during an eye screening.

2. The automated slit lamp system according to claim 1, further comprising a second computer program module configured for eye screening.

3. The automated slit lamp system according to claim 1, wherein the imaging optics comprise a plurality of objective lens and a plurality of tube lens.

4. The automated slit lamp system according to claim 1, wherein the imaging optics control is a 3-axis control, comprising:
a lead screw with roller on rails for z axis and y axis;
timing belts with rollers on rails for x axis;
at least one stepper motor for controlling movement of imaging optics along x axis, y axis and z axis, and
a stepper motor driver for controlling the at least one stepper motor.

5. The automated slit lamp system according to claim 1, wherein the first computer program module comprises a computer program for pupil detection and iris detection.

6. The automated slit lamp system according to claim 1, wherein the lighting optics comprises:
a LED light source;
a collector lens acting as a LED collector;
an adjustable mask for a spot or a slit for profiling light for an eye examination;
a field lens for creating a magnified intermediary image of aperture stop;
a condenser lens for condensing or collimating light, and
a beam splitter for making light coaxial with the imaging device and removing glares from an acquired image.

7. The automated slit lamp system according to claim 3, wherein the lighting optics further comprises a field diaphragm locating between the collector lens and the field lens.

8. The automated slit lamp system according to claim 3, wherein the lighting optics further comprises a brightness control aperture.

9. The automated slit lamp system according to claim 3, wherein the lighting optics further comprises a lens for posterior imaging.

10. The automated slit lamp system according to claim 1, wherein the lighting optics control comprises a stepper motor to rotate slit lighting at different degrees for controlling the lighting optics.

11. The automated slit lamp system according to claim 2, wherein the second computer program module for eye screening comprises a computer program for eye screening.

12. A method of eye screening with an automated slit lamp system with computer program parts, comprising:
conducting different eye examinations for detecting different eye conditions on each eye of a patient by using the automated slit lamp with computer program parts system of claim 1;
acquiring images from the different eye examinations;
processing the images by using the computer program pets to obtain gradings for the different eye examinations, and
generating a report of eye conditions that the patient is suspected of having by consolidating all the gradings from the different eye examinations.

13. The method of eye screening according to claim 12, wherein the different eye conditions are cataract, glaucoma, Age Related Macular Degeneration, Diabetic Retinopathy or other eye conditions.

14. The method of eye screening according to claim 13, wherein glaucoma is Narrow Angle Glaucoma or Open Angle Glaucoma.

15. The method of eye screening according to claim 12, wherein the method further comprises determining whether the images are gradable before processing the images to obtain gradings.

16. The method of eye screening according to claim 15, wherein the method further comprises adjusting focus, centering the images and positioning light at a correct position for adjusting the images as gradable images before processing the images to obtain gradings.
